(19) United States
(12) Patent Application Publication
(10) Pub. No.: US 2018/0016210 A1
(43) Pub. Date: Jan. 18, 2018

(54) COMPOUND BED DESIGN WITH ADDITIONAL REGENERATION STEPS FOR REMOVAL OF VARIOUS SULFUR SPECIES FROM LIGHTER HYDROCARBON STREAMS CONTAINING TRACE LEVELS OF OLEFINS

(71) Applicant: UOP LLC, Des Plaines, IL (US)

(72) Inventors: Bart J. T. Beuckels, Antwerp (BE); Bruno A. De Jonckheere, Antwerp (BE); Luk G. J. Verhulst, Antwerp (BE); Kris A. P. Eeman, Antwerp (BE); Fedrik K. Vancraeynest, Antwerp (BE)

(21) Appl. No.: 15/717,813

(22) Filed: Sep. 27, 2017

Related U.S. Application Data
(63) Continuation of application No. PCT/US2016/023666, filed on Mar. 23, 2016.
(60) Provisional application No. 62/139,514, filed on Mar. 27, 2015.

(51) Int. Cl. C07C 7/13; B01J 20/08; B01D 15/08; B01J 20/18; C07C 7/12; B01J 20/34

(52) U.S. Cl. CPC C07C 7/13; C07C 7/12; B01J 20/3483; B01J 20/3458; B01D 15/08; B01J 20/3408; B01J 20/18; B01J 20/08; B01J 20/3433

(57) ABSTRACT

A process is provided to remove impurities including water, mercaptans, carbonyl sulfide and hydrogen sulfide from hydrocarbon streams containing from 100 to 900 ppm light olefins. In the process, a compound bed containing multiple layers of molecular sieves is used to remove the specific impurities. In situations when the regeneration gas may contain sulfur compounds, a sulfur guard bed may be used to treat the regeneration gas prior to regenerating the compound adsorbent bed.

### CROSS-REFERENCE TO RELATED APPLICATIONS

This application is a Continuation of copending International Application No. PCT/US2016/023666 filed Mar. 23, 2016, which application claims priority from U.S. Provisional Application No. 62/139,514 filed Mar. 27, 2015, now expired, the contents of which cited applications are hereby incorporated by reference in their entirety.

### BACKGROUND OF THE INVENTION

The invention involves using different layers of adsorbents to remove impurities including water, mercaptans, carbonyl sulfide, hydrogen sulfide, disulfides and other sulfur components from a liquid hydrocarbon stream containing trace levels of olefins. Also the impact on the process cycle (regeneration) is described. The use of a sulfur guard bed on the regeneration gas in combination with these temperature swing adsorption (TSA) units is a further aspect of the invention.

The feed that is to be treated is a liquid hydrocarbon stream which consists mainly of propane and/or butane in addition to other lighter olefins. The invention is particularly relevant in processing liquid hydrocarbon streams that may contain olefins. These can be streams like propane, butane, liquid petroleum gas (LPG comprising a mixture of C3 and C4), and mixed C3+ (C3, C4 and C5+ together in a stream) as well as other streams.

The presence of olefins in the feed and/or the regeneration gas requires special attention due to the resulting malperformance of the adsorption unit as well as a serious reduction of the adsorbent unit's bed life if the effect of olefins is not fully taken into consideration when designing the unit. The current state of the art procedure does not take into consideration units handling hydrocarbon streams containing olefins in the ppm to hundreds of ppmv level.

Typical feeds to a liquid petroleum gas (LPG) treater in a natural gas plant do not contain olefins as the natural gas feed to the plant does not normally contain olefins. A typical feed to an olefin treater in a refinery can contain olefins, which, if present, are present in the % range. However, if the gas to the natural gas plant is mixed with a refinery off-gas, there often will be olefins in the LPG feed. There may be several hundreds of ppm's of propylene, butene and/or other lighter olefins such as 100-999 ppm, more often 100-750 ppm and most often about 100-500 ppm olefins. This is much lower than the volume in an olefin stream in a refinery, but much higher than what is found in natural gas from a well or in a typical pipeline.

This invention considers both the adsorption side and the regeneration side of the molecular sieve adsorption units of the present invention. The precautions that are necessary to meet the specification and to ensure performance of the unit during an acceptable period of time are described below.

### SUMMARY OF THE INVENTION

The invention provides a process for treating a liquid hydrocarbon stream comprising propane or butane, between about 10 ppm to 1000 ppmv light olefins and contaminants comprising sending the liquid hydrocarbon stream through a compound adsorbent bed comprising at least two layers of adsorbents to remove at least a portion of the contaminants.

The liquid hydrocarbon stream may comprises about 100 to 900 ppmv light olefins, 100 to 300 ppmv light olefins, 200 to 500 ppmv light olefins or other amount that is generally less than 1000 ppmv. The liquid hydrocarbon stream mainly comprises a mixture of C3, C4 and C5+ hydrocarbons as well as contaminants such as mercaptans, carbonyl sulfide, hydrogen sulfide, disulfides and water. A compound adsorbent bed is used to remove the contaminants that comprises molecular sieve adsorbents such as zeolites, alumina or a hybrid adsorbent comprising a mixture of zeolites and alumina. The composition of particular layers are selected to remove the particular contaminants that are present in the hydrocarbon stream. In one embodiment of the invention, the compound bed comprises a layer of zeolites to remove water, disulfides or light mercaptans and a layer of alumina to remove carbonyl sulfide or hydrogen sulfide. In another embodiment of the invention, the compound bed comprises a layer of a hybrid zeolite-alumina adsorbent for removal of water or light mercaptans, disulfides, carbonyl sulfide or hydrogen sulfide and a layer of alumina for removal of additional carbonyl sulfide and hydrogen sulfide. In yet another embodiment of the invention, the compound bed comprises a layer of molecular sieves to remove water, mercaptans and disulfides; and a layer of alumina to remove hydrogen sulfide and carbonyl sulfide. In another embodiment of the invention, the compound bed comprises a layer of hybrid alumina/zeolite to remove water, mercaptans and disulfides; and a layer of alumina to remove hydrogen sulfide and carbonyl sulfide.

The process of the invention may further comprise draining the hydrocarbon stream from the compound adsorbent bed and either pressurizing or depressurizing the adsorbent bed. In some embodiments of the invention, the process further comprises sending a cold purge gas through the compound adsorbent bed to remove a portion of light olefins from the adsorbent or sending a warm purge gas stream through the compound adsorbent bed to remove a majority of the light olefins. In some embodiments, after the higher temperature regeneration gas passes through the compound adsorbent bed, a cooler regeneration gas stream is sent through the compound adsorbent bed followed by pre-loading of the compound adsorbent bed with a low level of light olefins. In an embodiment of the invention where the hydrocarbon stream comprises about 100 to 300 ppm light olefins further comprising sending first a warm purge stream at about 90-110° C. through said compound adsorbent bed followed by sending a hot regeneration stream at about 200-300° C. through said compound adsorbent bed. The process further comprises sending a cool regeneration gas stream through the compound adsorbent bed to lower the temperature of the compound adsorbent bed. In some embodiments of the invention light olefins are injected into the cool regeneration gas stream to preload said compound adsorbent bed while further cooling the said compound adsorbent bed. This preloading step can only initiated when the bed is cool enough, e.g. at 90-110° C.

In some embodiments of the invention, the regeneration gas passes through a sulfur guard bed to remove sulfur impurities before it passes through the compound adsorbent bed.

When the regeneration gas comprises 5-50 ppmv or higher levels of sulfur compounds the gas may be treated to remove the sulfur compounds including mercaptans, carbonyl sulfide and hydrogen sulfide prior to passing through the compound adsorbent bed. In an embodiment of the invention, it will first be determined whether the regeneration gas comprises more than 5 ppmv sulfur compounds. When there is at least a low level of sulfur compounds in the gas stream, then it may be sent to a sulfur guard bed. In those instances when there are extremely low levels or undetectable levels of sulfur compounds then there is no need for the sulfur guard bed and it may be bypassed. The sulfur guard bed comprises a non-regenerative adsorbent and the sulfur guard bed may be a single bed, a system with two beds in series or a system with two beds in parallel.

### DETAILED DESCRIPTION OF THE INVENTION

The invention provides a process for treating a liquid hydrocarbon stream comprising propane or butane, between about 10 ppm to 1000 ppmv light olefins and contaminants comprising sending the liquid hydrocarbon stream through a compound adsorbent bed comprising at least two layers of adsorbents to remove at least a portion of the contaminants.

A compound bed using molecular sieves, including zeolites, alumina, other adsorbents and mixtures thereof may be used to treat a liquid hydrocarbon stream containing olefins to meet the low sulfur specification in the product.

The characteristics of the different adsorbents is used to meet the specification. Zeolites have a high capacity for removal of water, mercaptans and disulfides from natural gas liquid (NGL) streams. These adsorbents have been used to treat C₃/C₄/C₃₊ streams in many natural gas plants. Alumina have much higher capacity for removal of COS and/or H₂S from NGL streams than molecular sieves. They have been used to treat propane and butane stream with in natural gas plants. UOP hybrid AZ adsorbents which are a mixture of zeolite and activated alumina have a capacity for removal of water, mercaptans and disulfides as well as for removal of H₂S and COS. These adsorbent have been used in refineries to remove mercaptans, disulfides, COS and/or H₂S from streams with high olefin content.

After the adsorbent bed is saturated with sulfur, the bed undergoes a series of regeneration steps. Precautions may be required to account for the presence of olefins in the feed and/or the regeneration gas. These extra steps may include additional steps during the regeneration cycle and additional conditioning of the regeneration gas. Whether or not these precautions are required depends on the level of olefins in the feed and/or the type of adsorbent used in the compound bed as well as the level of olefins and H₂S in the regeneration gas.

In the practice of the present invention, a compound bed may be used. In one embodiment of the invention, this bed may consist of a zeolite layer for removal of water and/or lighter mercaptans and/or disulfides; and an alumina layer for removal of COS and/or H₂S. In another embodiment, the compound bed consists of a layer of a hybrid zeolite-alumina adsorbent for removal of water and/or lighter mercaptans and/or disulfides and a portion of the COS and/or H₂S and a separate layer of alumina for removal of additional ELS and/or COS. An alternate solution would involve a layer of hybrid zeolite/alumina adsorbent for removal of water and/or lighter mercaptans, disulfides, COS and/or H₂S and a separate layer of zeolites for removal of lighter mercaptans and/or disulfides.

In situations where olefin levels are as low as a few hundred ppmv of olefins, a recommended configuration is a compound bed of molecular sieves (removal of water, mercaptans and disulfides) and alumina (removal of H₂S and COS). In one particular potential project studied, the olefins content of the LPG feed is about 200-300 ppmv in the mixed LPG stream or less than 100-200 ppmv in each of a separated stream (C3 stream and C4 stream). When levels are significantly higher than a few hundreds of ppmv (but still below the high % levels of a refinery olefin stream), a recommended configuration is a compound bed of a hybrid alumina/zeolite adsorbent (removal of water, mercaptans) and alumina (removal of H₂S and COS).

In addition to treatment of the feed, there are special considerations to be taken in the regeneration cycle. After the bed is saturated with contaminants, the bed undergoes some of the following steps: Draining may be combined with pressurization or draining may be combined with depressurization (depending on regeneration gas pressure). There may be a cold or warm purge that may use stripping gas at ambient or intermediate temperature to strip off some of the olefins. Regeneration heating, using regeneration gas at higher temperature can be used to regenerate the beds by desorbing the contaminants from the adsorbent. Regeneration cooling, using regeneration gas at low temperature to cool down the bed. After the contaminants have been desorbed and the adsorption step is going to begin, there may be a pre-loading step by injecting olefins at low levels into the regeneration gas as soon as the beds are cool enough. There then may be a depressurization or repressurization step (depending on regeneration gas pressure).

Whether or not the warm purge step and/or the pre-loading are required depends on the level of olefins in the feed. In particular, when olefin levels in the feed are as low as a few hundred ppmv (100-300 ppm), it is recommended to have warm purge prior to a hot purge. When olefin levels are much higher than a few hundred ppmv (such as more than 100-300 ppm), it is recommended to have a warm purge prior to the hot purge and a preloading step before filling the bed with a feedstream. During the warm purge, the bed is at 90-110° C. which allows the coabsorbed olefins to be released before the bed is brought to 200-300° C. during the hot purge. During the preloading step, olefins are injected into the cool regeneration gas in order to pre-load the bed with olefins while it is further cooled. Only when the temperature of the bed is low enough (about 90-110° C.), the preloading step can be started, which is typically reached after several hours of cooling. Preloading the bed with olefins will reduce the temperature increase that results from filling the bed with the olefin rich liquid stream. The injection rate can be kept constant or it can be increased over time during the preloading step.

In some cases there needs to be additional treatment of the regeneration gas to insure that it is sulfur-free, in particular during cooling. A sulfur guard bed may be used to ensure that the regeneration gas to these adsorbent beds is free of sulfur. However, the use of a sulfur guard bed is valid for all adsorbent beds for liquid hydrocarbon stream whether or not their feeds contain olefins or not. The adsorbent beds that are using regenerative adsorbents for sulfur removal from streams such as C3/C4/C3+/LPG require the regeneration gas to be lean in sulfur to ensure the copper strip test (the corrosiveness of the hydrocarbon stream to copper in accordance with ASTM D130) and other quality specifications are achieved.

Typically, the residue gas from the NGL recovery unit is used to regenerate the adsorbent beds that treat the liquid hydrocarbons. In plants where ethane is recovered, the residue gas does not contain H₂S, since the H₂S goes with the ethane stream. However, in the case of plants where the C2+ is not recovered but instead is reinjected into the ground, the residue gas will contain ethane and thus also H₂S.

In that situation, there is a potential risk involved in loading the adsorbent bed with H₂S during regeneration. This situation may occur in particularly during the cooling step. When the bed is loaded with H₂S during cooling, the capacity of the adsorbent bed for removing H₂S in the next adsorption step will be reduced. Therefore, a sulfur-free regen gas needs to be available during cooling to meet the sulfur specification on the liquid product.

A sulfur guard bed can installed on the residue gas to ensure the regeneration gas is low in H₂S before being fed to the adsorbent bed, which contains one or different layers of adsorbents. The sulfur guard bed option is considered to be an economical solution when H₂S is in the 5-100 ppmv range. In addition, for higher H₂S levels and for other sulfur compound present in the residue gas, the sulfur guard bed option could be attractive.

The sulfur guard bed unit can be in continuous operation. Alternatively, it can only be in operation for a portion of each regeneration cycle, during a limited number of regeneration cycles or even during only one cycle. By running the sulfur guard bed unit on a non-continuous basis, the time between change-outs of the sulfur guard bed adsorbent can be increased significantly.

The sulfur guard bed unit will use a non-regenerative adsorbent, like e.g. a copper based adsorbent. The sulfur guard bed unit can be a single bed, a system with two beds in series (lead/lag) or two beds in parallel (2×100% or 2×50%).

There are several ways to operate the sulfur guard bed unit:

The sulfur guard bed unit can be in (semi-)continuous operation, while the regeneration gas is treated (most of the time). In this scenario, the residue gas is treated at least during the following steps: draining, warm purge, hot purge and cooling. The spent regeneration gas from the liquid treater is sent to battery limits (open loop regeneration).

In a second embodiment, the sulfur guard bed unit is only operated during the regeneration cooling step, while it is by-passed during the other steps of the regeneration cycle.

During each cooling step, fresh fuel gas is sent to the sulfur guard bed unit for processing. Next, the sulfur-free gas is sent to the liquid treater bed in cooling mode. The hot gas from the liquid treater is then sent to battery limits. (open loop regeneration—cooling only)

In a third embodiment, the sulfur guard bed unit is only removing H₂S from the residue gas during one or the first cycle. In the next cycles, the sulfur-free regeneration gas is recycled. This scheme will require a cooler and a blower. However, this option reduces the consumption of gas for regeneration of the liquid treaters (closed loop regeneration—cooling only).

1. A process for treating a liquid hydrocarbon stream comprising propane or butane, between about 10 ppm to 1000 ppmv light olefins and contaminants comprising sending said liquid hydrocarbon stream through a compound adsorbent bed comprising at least two layers of adsorbents to remove at least a portion of said contaminants.

2. The process of claim 1 wherein said liquid hydrocarbon stream comprises about 100 to 900 ppm light olefins.

3. The process of claim 1 wherein said liquid hydrocarbon stream comprises a mixture of C₃, C₄ and C₅+ hydrocarbons.

4. The process of claim 1 wherein said adsorbent bed comprises an adsorbent selected from the group consisting of zeolites, alumina and a hybrid adsorbent comprising a mixture of zeolites and alumina.

5. The process of claim 1 wherein said contaminants are selected from the group consisting of mercaptans, carbonyl sulfide, hydrogen sulfide, disulfides and water.

6. The process of claim 1 wherein said compound bed comprises a layer of zeolites to remove water, light mercaptans or disulfides and a layer of alumina to remove carbonyl sulfide or hydrogen sulfide.

7. The process of claim 1 wherein said compound bed comprises a layer of a hybrid zeolite-alumina adsorbent for removal of water, light mercaptans or disulfides, carbonyl sulfide or hydrogen sulfide and a layer of alumina for removal of additional carbonyl sulfide and hydrogen sulfide.

8. The process of claim 1 wherein said compound bed comprises a layer of molecular sieves to remove water, mercaptans and disulfides and a layer of alumina to remove hydrogen sulfide and carbonyl sulfide.

9. The process of claim 1 wherein said compound bed comprises a layer of hybrid alumina/zeolite to remove water, mercaptans and disulfides and a layer of alumina to remove hydrogen sulfide and carbonyl sulfide.

10. The process of claim 1 further comprising draining said hydrocarbon stream from said compound adsorbent bed and either pressurizing or depressurizing said adsorbent bed.

11. The process of claim 10 further comprising sending a cold purge gas through said compound adsorbent bed to remove a portion of light olefins from said adsorbent or sending a warm purge gas stream through said compound adsorbent bed to remove a majority of said light olefins.

12. The process of claim 10 wherein after said higher temperature regeneration gas passes through said compound adsorbent bed, a cooler regeneration gas stream is sent through said compound adsorbent bed

13. The process of claim 10 wherein after said higher temperature regeneration gas passes through said compound adsorbent bed, a cooler regeneration gas stream is sent through said compound adsorbent bed until it is cool enough to be pre-loaded followed by the actual pre-loading of said compound adsorbent bed with a low level of said light olefins.

14. The process of claim 1 wherein said hydrocarbon stream comprises about 100 to 300 ppm light olefins further comprising sending first a warm purge stream at about 90° to 110° C. through said compound adsorbent bed followed by sending a hot regeneration stream at about 200° to 300° C. through said compound adsorbent bed.

15. The process of claim 11 further comprising sending a cool regeneration gas stream through said compound adsorbent bed to lower a temperature of said compound adsorbent bed to about 90° to 110° C.

16. The process of claim 15 followed by a process wherein olefins are injected in said cool regeneration gas stream to further cool and preload said compound adsorbent bed to reduce a temperature increase upon sending said hydrocarbon stream to said compound adsorbent bed.

17. The process of claim 11 wherein said cold or warm purge gas passes through a sulfur guard bed to remove sulfur impurities before said cold or warm purge gas passes through said compound adsorbent bed.

18. The process of claim 17 wherein said cold or warm purge gas comprises 5-100 ppmv sulfur compounds prior to passing through said compound adsorbent bed.

19. The process of claim 11 further comprising determining whether said cold or warm purge gas comprises more than 5 ppmv sulfur compounds and then sending said cold or warm purge gas to a sulfur guard bed when more than 5 ppmv sulfur compounds are determined to be in said cold or warm purge gas stream.

20. The process of claim 12 wherein said cool regeneration gas passes through a sulfur guard bed to remove sulfur impurities before said cool regeneration gas passes through said compound adsorbent bed.

21. The process of claim 20 wherein said cool regeneration gas comprises 5-100 ppmv sulfur compounds prior to passing through said compound adsorbent bed.

22. The process of claim 12 further comprising determining whether said cool regeneration gas comprises more than 5 ppmv sulfur compounds and then sending said cool regeneration gas to a sulfur guard bed when more than 5 ppmv sulfur compounds are determined to be in said cool regeneration stream.

23. The process of claim 17 wherein said sulfur guard bed comprises a non-regenerative adsorbent.

24. The process of claim 20 wherein said sulfur guard bed comprises a non-regenerative adsorbent.

25. The process of claim 17 wherein said sulfur guard bed is a single bed, a system with two beds in series or a system with two beds in parallel.

26. The process of claim 20 wherein said sulfur guard bed is a single bed, a system with two beds in series or a system with two beds in parallel.
